(19) United States
(12) Patent Application Publication
(10) Pub. No.: US 2025/0213207 A1
(43) Pub. Date: Jul. 3, 2025

(54) OPERATION IMAGE ALIGNMENT METHOD AND SYSTEM THEREOF

(71) Applicant: METAL INDUSTRIES RESEARCH & DEVELOPMENT CENTRE, KAOHSIUNG (TW)

(72) Inventors: Yu-Wen CHENG, Kaohsiung City (TW); Jin-Yuan SYUE, Tainan City (TW); Bing-Feng HUANG, Kaohsiung City (TW); Chin Fang HSIEH, Kaohsiung City (TW)

(21) Appl. No.: 18/398,438

(22) Filed: Dec. 28, 2023

(51) Int. Cl. A61B 6/46; A61B 6/00; A61B 6/58; A61B 90/00

(52) U.S. Cl. CPC A61B 6/463; A61B 6/4441; A61B 6/582; A61B 90/36; A61B 2090/364

(57) ABSTRACT

An operation image alignment method includes: inputting an actual three-dimensional image and an actual two-dimensional image of an actual desired part; converting the actual three-dimensional image into multiple sets of two-dimensional projection images according to image parameters of the actual three-dimensional image through an image alignment prediction model; comparing each of the sets of two-dimensional projection images to the actual two-dimensional image to calculate an image parameter difference value for each of the sets of two-dimensional projection image, and selecting one of the sets of two-dimensional projection image to obtain a predicted rotation angle and translation. The one of the sets of two-dimensional projection image has an image parameter difference value matching a preset difference value. The multiple sets of historical images containing at least one historical three-dimensional image and at least one historical two-dimensional image are used as a training data set of the image alignment prediction model.

### BACKGROUND

### Technical Field

The invention relates to operation image alignment methods and systems, and particularly related to an operation image alignment method and system for aligning three-dimensional images and two-dimensional images.

### Description of Related Art

The operation navigation system is an extremely important part in medical operation, assisting doctors to accurately operate on the lesion location during the operation. Before performing an operation, a computed tomography (CT) scanner or a magnetic resonance imaging (MRI) scanner is used to obtain three-dimensional images of an operation site, allowing doctors to accurately grasp the image contents of the lesion location. When performing the operation, registration of the operation images is required. The lesion locations in two-dimensional image (obtained during the operation) and the three-dimensional images (obtained before the operation) are aligned for dynamic image tracking by the subsequent navigation system. Therefore, reducing alignment error and alignment processing time of operation images is a concern matter to those skilled in the art.

### SUMMARY

An operation image alignment method performed by a computer system, comprising: inputting an actual three-dimensional image and an actual two-dimensional image of an actual desired part into the computer system; converting the actual three-dimensional image into multiple sets of two-dimensional projection image according to image parameters of the actual three-dimensional image through a built-in image alignment prediction model of the computer system; comparing each of the sets of two-dimensional projection image to the actual two-dimensional image to calculate an image parameter difference value for each of the sets of two-dimensional projection image, and selecting one of the sets of two-dimensional projection image to obtain a predicted rotation angle and a predicted translation, wherein the one of the sets of two-dimensional projection image has an image parameter difference value matching a preset difference value; the image alignment prediction model is an artificial intelligence model trained by a model algorithm, and multiple sets of historical images including at least one historical three-dimensional image and at least one historical two-dimensional image are used as a training data set of the image alignment prediction model.

In some embodiments, the operation image alignment method further comprising a model building step to build the image alignment prediction model, which comprises: defining historical desired regions of an historical desired part from the at least one historical three-dimensional image and the at least one historical two-dimensional image of each set of historical images, in which each of the historical desired regions includes a historical position information.

In some embodiments, the model building step further comprising: converting the at least one historical three-dimensional image of each set of historical images into at least one historical two-dimensional projection image with a first perspective or a second perspective through an image projection transformation technology; and using the historical position information of the historical desired regions as an initial position, and obtaining a historical rotation angle and a historical translation in the first perspective or the second perspective between the at least one historical three-dimensional image and the at least one historical two-dimensional image in each set of historical images through the at least one historical two-dimensional projection image.

In some embodiments, the first perspective is a side-view, the second perspective is a top-view.

In some embodiments, the image parameters include an image contour and an image gradient value.

In some embodiments, the model algorithm is one of a generative adversarial networks algorithm and a deep iterative 2D/3D registration algorithm.

In some embodiments, the operation image alignment method further comprising: using an imaging equipment to capture the actual two-dimensional image of the actual desired part, in which the actual two-dimensional image includes an actual position information.

An operation image alignment system which is to use a computer system to perform the operation image alignment method as described in any of the foregoing.

In some embodiments, the operation image alignment system further comprises an imaging equipment, which is a C-arm X-ray machine with a transmitter and a receiver.

In some embodiments, the operation image alignment system further comprises a computed tomography equipment, which is used to capture the actual desired part to obtain the actual three-dimensional image.

### BRIEF DESCRIPTION OF THE DRAWINGS

The aspect of the invention can be better understood from the following detailed description combined with the accompanying drawings. It should be noted that features are not drawn to scale in accordance with standard industry practice. In fact, the dimensions of each feature may be arbitrarily increased or decreased for clarity of discussion.

FIG. 1 is a schematic diagram of the operation image alignment system according to an embodiment of the invention.

FIG. 2 is a schematic diagram of the operation image alignment method according to an embodiment of the invention.

FIG. 3A and FIG. 3B are schematic diagrams of desired regions in the historical two-dimensional images of side-view images and top-view images respectively according to an embodiment of the invention.

FIG. 4 is a schematic diagram of the historical two-dimensional projection image according to an embodiment of the invention.

### DETAILED DESCRIPTION

Embodiments of the present invention are discussed in detail below. However, it should be appreciated that the embodiments provide many applicable concepts that can be embodied in variety of specific contexts. The embodiments discussed and disclosed are for illustration only and are not intended to limit the scope of the invention.

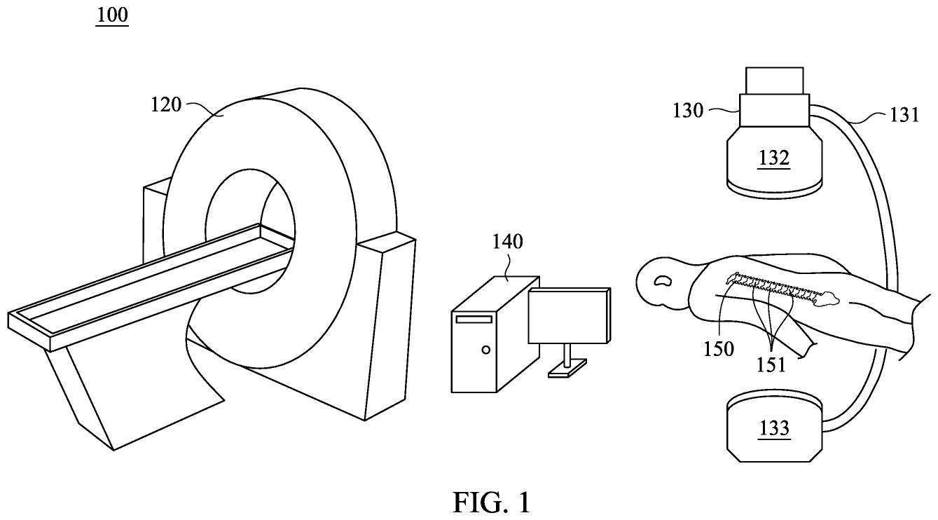

FIG. 1 is a schematic diagram of the operation image alignment system 100 according to an embodiment of the invention. The operation image alignment system 100 includes a three-dimensional imaging equipment 120, a two-dimensional imaging equipment 130 and a computer system 140. The operation image alignment system 100 uses the computer system 140 to train an artificial intelligence model to align the three-dimensional images (captured through the three-dimensional imaging equipment 120 before operation) with the two-dimensional images (captured through the two-dimensional imaging equipment 130 during operation) for a desired part. In this way, not only the alignment error of operation images can be reduced, but also the time required to complete the registration of operation images during the operation can be reduced. In the following embodiments, a spine 150 is used as the desired part, and vertebrae 151 in the spine 150 are used as the desired regions. It should be understood that other parts requiring regional alignment and operation navigation are within the scope of the invention.

The three-dimensional imaging equipment 120 is used to capture the three-dimensional images before operation. The three-dimensional imaging equipment 120 can be a magnetic resonance imaging (MRI) scanner, a computed tomography (CT) scanner, a positron emission tomography (PET) scanner, a single photon emission CT (SPECT) scanner, or any device that can obtain the three-dimensional images of a target. For example, patients can take computed tomography images of the spine 150 before the operation, and convert the spine 150 into a three-dimensional simulated object through the computer system 140. Therefore, a three-dimensional image with a three-dimensional simulated object can be obtained, and the at least one vertebra 151 is separate therefrom.

The two-dimensional imaging equipment 130 is used to capture the two-dimensional images during operation. The two-dimensional imaging equipment 130 is a C-arm X-ray machine with a C-frame 131, a transmitter 132 and a receiver 133. The C-frame 131 drives the transmitter 132 and the receiver 133 to rotate around the target, so that the C-arm X-ray machine can capture the two-dimensional images of the target at different angles. For example, the transmitter 132 and the receiver 133 are disposed opposite to each other, the transmitter 132 emits X-rays to the vertebrae 151, and the receiver 133 receives the X-rays passing through the vertebrae 151, thereby converting the X-rays into the two-dimensional images. The two-dimensional images include images taken from the front and back (A-P view) (i.e., top-view) and images taken from the side (lateral view) (i.e., side-view).

The computer system 140 is communicatively connected to the three-dimensional imaging equipment 120 and the two-dimensional imaging equipment 130, and data can be transmitted between each other through any wired or wireless method. The computer system 140 includes memory and processor, which can used to store multiple sets of historical images (each set of historical images includes a historical three-dimensional image I_{H1} and a corresponding historical two-dimensional image I_{H2}), and used to perform image processing on these historical images to define at least one vertebra 151 from these historical images. Then, the historical three-dimensional images I_{H1} and the historical two-dimensional images I_{H2} are used as a training data set to train an artificial intelligence model, thereby building the image alignment prediction model of the invention. Among them, the training of the image alignment prediction model is achieved through an image projection conversion technology, which converts the historical three-dimensional image I_{H1} into historical two-dimensional projection images I_{PH1}. Therefore, the relative position information between each the historical two-dimensional projection image I_{PH1} and the historical two-dimensional image I_{H2} with different perspectives can be obtained. In particular, each of the desired vertebrae 151 in the actual three-dimensional image I_{T1} and the actual two-dimensional image I_{T2} can be aligned together through the image alignment prediction model while the computer system 140 receives the actual three-dimensional image I_{T1} (captured through the three-dimensional imaging equipment 120 before operation) and the actual two-dimensional image I_{T2} (captured through the two-dimensional imaging equipment 130 during the operation), allowing doctors to perform subsequent operational image tracking. The computer system 140 can be a smartphone, a tablet, a personal computer, a notebook computer, a server, an industrial computer or other electronic devices with computing capabilities, and the invention is not limited thereto.

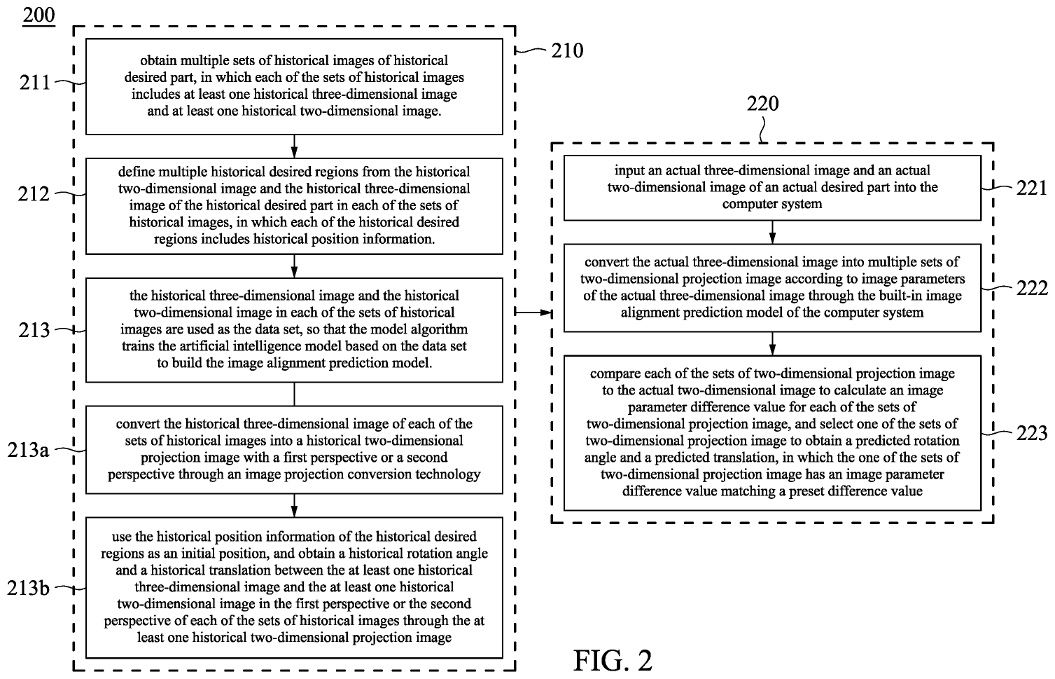

FIG. 2 is a schematic diagram of the operation image alignment method 200 according to an embodiment of the invention. The operation image alignment method 200 includes a model building step 210 and an online aligning step 220. The model building step 210 includes steps 211-213, and the step 213 further includes steps 213a and 213b. The online aligning step 220 includes steps 221 and 213. The operation image alignment method 200 may be applied to the operation image alignment system 100 shown in FIG. 1 or other similar structures. The following takes the operation image alignment method 200 and the operation image alignment system 100 shown in FIG. 1 as an example for description.

In the model building step 210, step 211 is performed first to obtain multiple sets of historical images of historical spine 150. Each set of historical images includes at least one historical three-dimensional image I_{H1} of the historical spine 150 (captured through the three-dimensional imaging equipment 120 in the past) and at least one historical two-dimensional image I_{H2} of the historical spine 150 (captured through the two-dimensional imaging equipment 130 in the past). The sources of the historical spines 150 can be taken from different people, and the historical two-dimensional image I_{H2} and the historical three-dimensional image I_{H1} captured in the past are used as the training data set for training the artificial intelligence model. In some embodiments, one historical three-dimensional image I_{H1} corresponds to one or more historical two-dimensional images I_{H2} with different perspective. For example, but not limited to, the historical two-dimensional image I_{H2} is an image captured from a side-view (a first perspective), or an image captured from a top-view (a second perspective).

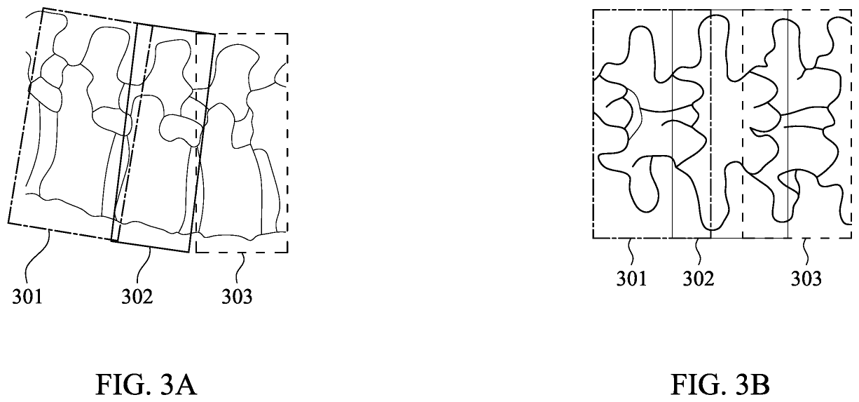

In step 212, multiple historical desired regions (corresponding to multiple historical desired vertebrae 151) are defined from the historical two-dimensional image I_{H2} and the historical three-dimensional image I_{H1} of the historical spine 150 in each set of historical images. Please referring to FIG. 3A and FIG. 3B, the multiple historical desired regions in the side-view image (FIG. 3A) and the top-view (FIG. 3B) of the historical two-dimensional images I_{H2} include region 301, region 302 and region 303, which include each of the historical desired vertebrae 151 respectively. Each defined historical desired regions includes historical position information, where the historical position information is the position information of each historical desired region in three-dimensional space.

Please return to FIG. 2. In step 213, the historical three-dimensional image I_{H1} and the historical two-dimensional image I_{H2} in each set of historical images are used as the training data set, so that the model algorithm trains the artificial intelligence model based on the training data set to build the image alignment prediction model. In this way, the image alignment prediction model can predict a correct rotation angle and a correct translation based on the actual three-dimensional image I_{T1} and the actual two-dimensional image I_{T2} of each actual desired vertebra 151 in the actual spine 150, thereby enabling the actual three-dimensional images I_{T1} of the actual desired vertebrae 151 are aligned with the actual two-dimensional images I_{T2} one by one. In some embodiments, model algorithms for training the image alignment prediction model include, but are not limited to, a generative adversarial networks algorithm and a deep iterative 2D/3D registration algorithm.

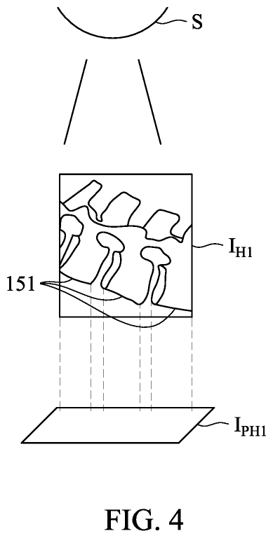

Referring to FIG. 2. Step 213 in the embodiment of the invention further includes step 213a and step 213b. In step 213a, the computer system 140 uses image projection conversion technology to convert the historical three-dimensional image I_{H1} of each set of historical images into a historical two-dimensional projection image I_{PH1} with a first perspective or a second perspective. The Image projection conversion technologies include but not limited to perspective transformation, or any technology that can convert a three-dimensional image into a two-dimensional image. As shown in FIG. 4, the first perspective and the second perspective are respectively the side-view and the top-view of the historical two-dimensional projection image I_{PH1} obtained by projecting a radioactive source S onto the historical three-dimensional image I_{H1}. Alternatively, the historical three-dimensional image I_{H1} can be projected through the radiation source S to obtain the historical two-dimensional projection image I_{PH1} with any perspective. Among them, the historical three-dimensional image I_{H1} includes multiple desired vertebrae 151 of the historical spine 150, which is converted into a historical two-dimensional projection image I_{PH1} with the first perspective or the second perspective through the image projection conversion technology. After that, multiple historical desired vertebrae 151 are cut out one by one from the historical two-dimensional projection image I_{PH1}. Alternatively, the computer system 140 cuts out multiple historical desired vertebrae 151 from the historical three-dimensional image I_{H1} as multiple historical three-dimensional images I_{H1} first, and then convert them one by one into multiple historical two-dimensional projection images I_{PH1} with the first perspective or the second perspective through the image projection conversion technology, the invention is not limited thereto.

Returning to FIG. 2, in step 213b, the historical position information of each historical desired region are used as an initial position, and the historical correct rotation angle and the historical correct translation between the at least one historical three-dimensional image I_{H1} and the at least one historical two-dimensional image I_{H2} in the first perspective or the second perspective of each set of historical images are obtained through the historical two-dimensional projection image I_{PH1}. Specifically, the historical position information of the historical desired region (for example, the desired vertebra 151) in the historical two-dimensional image I_{H2} and the historical three-dimensional image I_{H1} can be used as the initial position in the coordinate system for the computer system 140 to obtain the image parameter difference between the historical two-dimensional projection image I_{PH1} and the historical two-dimensional image I_{H2}. Therefore, the historical correct rotation angle and the historical correct translation between the historical three-dimensional image I_{H1} and the historical two-dimensional image I_{H2} in the top-view or side-view can be obtained based on the image parameter difference, so that the historical desired vertebrae 151 in the historical two-dimensional image I_{H2} can be aligned with the desired vertebrae 151 in the historical three-dimensional images I_{H1} one by one. In other word, the historical correct rotation angle and the historical correct translation allow the image alignment prediction model to rotate/translate the historical three-dimensional image I_{H1} of the desired vertebra 151, so that it can be aligned to the historical desired vertebra 151 in the historical two-dimensional image I_{H2}. If there are multiple historical desired vertebrae 151, multiple historical three-dimensional images I_{H1} corresponding to these historical desired vertebrae 151 can be rotated/translated, so that these historical three-dimensional images I_{H1} can be aligned to the historical vertebrae 151 in the historical two-dimensional image I_{H2} one by one.

Referring to FIG. 2, after performing the model building step 210, the online alignment step 220 is performed. First, step 221 is performed to input the actual three-dimensional image I_{T1} and the actual two-dimensional image I_{T2} of the actual spine 150 into the computer system 140. Then in step 222, the computer system uses the image alignment prediction model established in the model building step 210 to generate multiple sets of two-dimensional projection images I_{PT1}. Among them, multiple sets of two-dimensional projection images are generated by converting the actual three-dimensional images I_{T1} through the aforementioned image projection conversion technology based on various image parameters. Each set of two-dimensional projection images I_{PT1} is a two-dimensional projection image from any perspective obtained by projecting the simulated radioactive source S to the actual three-dimensional image I_{T1} in the image alignment prediction model. In embodiments of the invention, the image parameters include but are not limited to image contours and image brightness variation values (i.e., image gradient values, used to indicate the degree of light and shade changes in the images).

Then in step 223, the image alignment prediction model compares each of the sets of two-dimensional projection images I_{PT1} to the actual two-dimensional image I_{T2}, and calculates the image parameter difference value between each of the sets of two-dimensional projection images I_{PT1} and the actual two-dimensional image I_{T2}. Afterwards, the image alignment prediction model selects one of the sets of two-dimensional projection images I_{PT1} whose image parameter difference value matching the preset difference value as a prediction result, thereby obtaining the predicted rotation angle and the predicted translation between the desired vertebra 151 in the actual three-dimensional image I_{T1} and the actual two-dimensional image I_{T2}. In this way, the operation navigation system can complete the registration of the operation images based on the alignment between the actual three-dimensional image I_{T1} and the actual two-dimensional image I_{T2}.

In some embodiments, the model building step 210 further includes a discrimination step of the image alignment prediction model to train the accuracy of the image alignment prediction model. Specifically, the computer system 140 inputs an experimental three-dimensional image I_{E1} and an experimental two-dimensional image I_{E2} of the historical spine 150 into the image alignment prediction model, thereby obtaining an experimental predicted rotation angle and an experimental predicted translation between the experimental three-dimensional image I_{E1} and the experimental two-dimensional image I_{E2}. Then the image alignment prediction model generates the experimental predicted projection image PE based on the experimental predicted rotation angle and the experimental predicted translation. Next, the generated experimental prediction projection image PE is used to build an image alignment discriminator model to determine whether the accuracy of the image alignment prediction model is qualified. Specifically, each of the experimental three-dimensional images I_{E1} and the experimental two-dimensional images I_{E2} are input into the image alignment prediction model as an experimental set in the training data set; comparing the experimental predicted projection image PE (which includes the experimental predicted rotation angle and experimental predicted translation between the experimental three-dimensional image I_{E1} and the experimental two-dimensional image I_{E2}) predicted by the image alignment prediction model with the correct experimental two-dimensional image I_{E2}; and training the artificial intelligence model to build the image alignment discriminator model. Therefore, when the image alignment discriminator model cannot determine the authenticity between the experimental predicted projection image PE and the correct experimental two-dimensional image I_{E2}, the prediction of the image alignment prediction model is deemed to be accurate enough. Otherwise, the image alignment prediction model is re-predicted until the accuracy of the prediction result is qualified.

During the entire alignment processes, the image alignment prediction model uses the actual position information of the actual desired vertebra 151 in the actual three-dimensional image I_{T1} and the actual two-dimensional image I_{T2} as the initial position, and uses the image alignment discriminator model to continuously determine whether the prediction results of the image alignment model are accurate enough until the correct rotation angle and the correct translation between the actual three-dimensional image I_{T1} and the actual two-dimensional image I_{T2} are predicted. In this way, the actual three-dimensional image and the actual two-dimensional image of the actual desired vertebra 151 can finally be aligned with each other. It should be understood that establishing the image alignment discriminator model is only to improve the prediction accuracy of the image alignment prediction model. In fact, in some embodiments of the invention, even if the establishment of the image alignment discriminator model is omitted, it will not affect the image alignment between the three-dimensional images and the two-dimensional images of the invention.

In addition, through continuous prediction and adjustment, the image alignment model can perform a series of alignment processes, so that the actual three-dimensional image I_{T1} of the desired vertebra 151 can finally be aligned with the actual two-dimensional images I_{T2} of the top-view and the side-view.

The features of several embodiments are described above, so that those skilled in the art can better understand the aspects of the invention. Those skilled in the art should understand that they can easily use the invention as a basis to design or modify other processes and structures to achieve the same goals and/or achieve the same advantages as the embodiments. Those skilled in the art should also understand that these equivalent structures do not deviate from the spirit and scope of the invention, and they can make various changes, substitutions and variations without departing from the spirit and the scope of the invention.

What is claimed is:

1. An operation image alignment method performed by a computer system comprising:
inputting an actual three-dimensional image and an actual two-dimensional image of an actual desired part into the computer system;
converting the actual three-dimensional image into multiple sets of two-dimensional projection image according to image parameters of the actual three-dimensional image through a built-in image alignment prediction model of the computer system; and
comparing each of the sets of two-dimensional projection image to the actual two-dimensional image to calculate an image parameter difference value for each of the sets of two-dimensional projection image, and selecting one of the sets of two-dimensional projection image to obtain a predicted rotation angle and a predicted translation, wherein the one of the sets of two-dimensional projection image has an image parameter difference value matching a preset difference value;
wherein the image alignment prediction model is an artificial intelligence model trained by a model algorithm, and multiple sets of historical images comprising at least one historical three-dimensional image and at least one historical two-dimensional image are used as a training data set of the image alignment prediction model.

2. The operation image alignment method of claim 1, further comprising a model building step to build the image alignment prediction model, which comprises:
defining historical desired regions of an historical desired part from the at least one historical three-dimensional image and the at least one historical two-dimensional image of each set of historical images, wherein each of the historical desired regions includes a historical position information.

3. The operation image alignment method of claim 2, wherein the model building step further comprising:
converting the at least one historical three-dimensional image of each of the sets of historical images into at least one historical two-dimensional projection image with a first perspective or a second perspective through an image projection conversion technology; and
using the historical position information of the historical desired regions as an initial position, and obtaining a historical rotation angle and a historical translation in the first perspective or the second perspective between the at least one historical three-dimensional image and the at least one historical two-dimensional image in each of the sets of historical images through the at least one historical two-dimensional projection image.

4. The operation image alignment method of claim 3, wherein the first perspective is a side-view, the second perspective is a top-view.

5. The operation image alignment method of claim 1, wherein the image parameters include an image contour and an image gradient value.

6. The operation image alignment method of claim 1, wherein the model algorithm is one of a generative adversarial networks algorithm and a deep iterative 2D/3D registration algorithm.

7. The operation image alignment method of claim 1, further comprising:
using an imaging equipment to capture the actual two-dimensional image of the actual desired part, wherein the actual two-dimensional image includes an actual position information.

8. An operation image alignment system, wherein the operation image alignment system is to use a computer system to perform the operation image alignment method as claimed in claim 1.

9. An operation image alignment system, wherein the operation image alignment system is to use a computer system to perform the operation image alignment method as claimed in claim 2.

10. An operation image alignment system, wherein the operation image alignment system is to use a computer system to perform the operation image alignment method as claimed in claim 3.

11. An operation image alignment system, wherein the operation image alignment system is to use a computer system to perform the operation image alignment method as claimed in claim 4.

12. An operation image alignment system, wherein the operation image alignment system is to use a computer system to perform the operation image alignment method as claimed in claim 5.

13. An operation image alignment system, wherein the operation image alignment system is to use a computer system to perform the operation image alignment method as claimed in claim 6.

14. An operation image alignment system, wherein the operation image alignment system is to use a computer system to perform the operation image alignment method as claimed in claim 7.

15. The operation image alignment system of claim 8, further comprising an imaging equipment, wherein the imaging equipment is a C-arm X-ray machine with a transmitter and a receiver.

16. The operation image alignment system of claim 8, further comprising:
a computed tomography equipment used to capture the actual desired part to obtain the actual three-dimensional image.
